(19) United States
(12) Patent Application Publication
(10) Pub. No.: US 2026/0077195 A1
(43) Pub. Date: Mar. 19, 2026

(54) COCHLEAR IMPLANTS HAVING MRI-COMPATIBLE MAGNET ASSEMBLIES AND ASSOCIATED SYSTEMS AND METHODS

(71) Applicant: Advanced Bionics, LLC, Valencia, CA (US)

(72) Inventors: Daniel Alexander Alanis, Newhall, CA (US); Stephan Geiger, Ventura, CA (US); James George Elcoate Smith, Santa Clarita, CA (US); Nicholas Anthony Wise, Pasadena, CA (US)

(21) Appl. No.: 18/885,649

(22) Filed: Sep. 14, 2024

(51) Int. Cl. A61N 1/36; A61N 1/05

(52) U.S. Cl. CPC A61N 1/36038; A61N 1/0541

(57) ABSTRACT

A magnet assembly including a case defining a central axis, a magnet frame within the case and rotatable about the central axis of the case, and first, second and third elongate magnets that are located within the frame, with the third elongate magnet located between the first and second elongate magnets, and that each define a longitudinal axis and a N-S direction that is perpendicular to the longitudinal axis. The first and third elongate magnets may be separated from one another by a first fixed non-zero distance that is perpendicular to at least one of their longitudinal axes, the second and third elongate magnets may be separated from one another by a second fixed non-zero distance that is perpendicular to at least one of their longitudinal axes, at least one of the elongate magnets is rotatable about its longitudinal axis relative to the frame, and at least one of the elongate magnets is not rotatable about its longitudinal axis relative to the frame.

### BACKGROUND

### 1. Field

The present disclosure relates generally to implantable cochlear stimulation (or “ICS”) systems.

### 2. Description of the Related Art

ICS systems are used to help the profoundly deaf perceive a sensation of sound by directly exciting the intact auditory nerve with controlled impulses of electrical current. Ambient sound pressure waves are picked up by an externally worn microphone and converted to electrical signals. The electrical signals, in turn, are processed by a sound processor, converted to a pulse sequence having varying pulse widths, rates and/or amplitudes, and transmitted to an implanted receiver circuit of the ICS system. The implanted receiver circuit is connected to an implantable electrode array that has been inserted into the cochlea of the inner ear, and electrical stimulation current is applied to varying electrode combinations to create a perception of sound. The electrode array may, alternatively, be directly inserted into the cochlear nerve without residing in the cochlea. A representative ICS system is disclosed in U.S. Pat. No. 5,824,022, which is entitled “Cochlear Stimulation System Employing Behind-The-Ear Sound processor With Remote Control” and incorporated herein by reference in its entirety. Examples of commercially available ICS sound processors include, but are not limited to, the Naida™ CI M Series sound processor, the Naida™ CI Q Series sound processor and the Neptune™ body worn sound processor, which are available from Advanced Bionics.

As alluded to above, some ICS systems include an implantable cochlear stimulator (or “cochlear implant”), a sound processor unit (e.g., a body worn processor or behind-the-ear processor), and a microphone that is part of, or is in communication with, the sound processor unit. The cochlear implant communicates with the sound processor unit and, some ICS systems include a headpiece that is in communication with both the sound processor unit and the cochlear implant. The headpiece communicates with the cochlear implant by way of a transmitter (e.g., an antenna) on the headpiece and a receiver (e.g., an antenna) on the implant. Optimum communication is achieved when the transmitter and the receiver are aligned with one another. To that end, the headpiece and the cochlear implant may include respective positioning magnets that are attracted to one another, and that maintain the position of the headpiece transmitter over the implant receiver. The implant magnet may, for example, be located within a pocket in the cochlear implant housing. The skin and subcutaneous tissue that separates the headpiece magnet and implant magnet is sometimes referred to as the “skin flap,” which is frequently 3 mm to 11 mm thick, and is sometimes thicker.

The present inventors have determined that conventional cochlear implants and stimulation systems are susceptible to improvement. For example, the magnet in some conventional cochlear implant is a disk-shaped axially magnetized magnet that has north and south magnetic dipoles which are aligned in the axial direction of the disk. Such magnets are not compatible with magnetic resonance imaging (“MRI”) systems, and may have to be surgically removed from the cochlear implant prior to the MRI procedure and then surgically replaced thereafter. Other cochlear implants include a diametrically magnetized disk-shaped magnet that is rotatable relative to the remainder of the implant about its central axis, and that has a N-S orientation which is perpendicular to the central axis. The present inventors have determined that diametrically magnetized disk-shaped magnets are less than optimal because a dominant magnetic field, such as the MRI magnetic field, that is misaligned by at least 30° or more from the N-S direction of the magnet may demagnetize the magnet or generate an amount of torque on the magnet that is sufficient to dislodge or reverse the magnet and/or dislocate the associated cochlear implant and/or cause excessive discomfort to the patient.

More recently, cochlear implants with MRI-compatible magnet apparatus (or “magnet assemblies”) have been introduced. The MRI-compatible magnet apparatus have a case defining a central axis, a frame within the case that is rotatable relative to the case about the central axis, and two or more elongate diametrically magnetized magnets that are located in the frame in close proximity to one another and that are rotatable about their respective longitudinal axis relative to the frame. This combination allows the magnets to align with three-dimensional (3D) MRI magnetic fields, regardless of field direction, which results in very low amounts of torque on the magnets. Examples of such MRI-compatible magnet apparatus may be found in U.S. Pat. Nos. 9,919,154, 10,463,849, and 10,532,209. Another proposed magnet apparatus, which includes a single elongate magnet, is described in PCT Pat. Pub. No. 2020/092185 A1.

Although such MRI-compatible magnet apparatus have proven to be a significant advance in the art, the present inventors have determined that they are susceptible to improvement. For example, relatively thick skin flaps (e.g., skin flaps of at least 12 mm) create a relatively large distance between the headpiece magnet and the rotatable magnets of MRI-compatible magnet apparatus. In the exemplary context of an MRI-compatible magnet apparatus that has only two (i.e., two and no more than two) rotatable elongate diametrically magnetized magnets and is used in conjunction with an axially magnetized headpiece magnet, the present inventors have determined that, due to the relatively weak attraction force between the headpiece magnet and implant magnets that is associated with the relatively large distance, the magnetic attraction force between the implant magnets may prevent the implant magnets from rotating into optimal alignment with the magnetic field of the headpiece magnet. In addition to reducing the headpiece retention force provided by the magnets, non-optimal alignment of the magnetic fields can result in misalignment of the headpiece and implant magnets and, accordingly, misalignment of the headpiece and implant antennas. Turning to MRI-compatible magnet apparatus that have only one (i.e., one and no more than one) rotatable elongate diametrically magnetized magnet and are used in conjunction with an axially magnetized headpiece magnet, the present inventors have determined that there is a relatively weak attraction force between the headpiece magnet and single implant magnet.

### SUMMARY

A magnet assembly in accordance with at least one of the present inventions may include a case defining a central axis, a magnet frame within the case and rotatable about the central axis of the case, and first, second and third elongate magnets that are located within the frame, with the third elongate magnet located between the first and second elongate magnets, and that each define a longitudinal axis and a N-S direction that is perpendicular to the longitudinal axis. The first and third elongate magnets may be separated from one another by a first fixed non-zero distance that is perpendicular to at least one of their longitudinal axes, the second and third elongate magnets may be separated from one another by a second fixed non-zero distance that is perpendicular to at least one of their longitudinal axes, at least one of the elongate magnets is rotatable about its longitudinal axis relative to the frame, and at least one of the elongate magnets is not rotatable about its longitudinal axis relative to the frame. Alternatively, in some implementations where the first and second magnets are not rotational, they may be other than elongate magnets with a longitudinal axes.

A method in accordance with at least one of the present inventions may include removing a magnet or a magnet assembly from an implanted cochlear implant and installing the magnet assembly in the preceding paragraph in place of the removed magnet or magnet assembly. A system in accordance with at least one of the present inventions may include a head wearable external component, including an axially magnetized external magnet, and a cochlear implant having a cochlear lead, an implant antenna, an implant processor and the magnet assembly in the preceding paragraph.

There are a number of advantages associated with such apparatus and methods. By way of example, but not limitation, in some implementations, the magnetic force associated with a non-rotatable third elongate magnet offsets some of the magnetic attraction force between rotatable first and second elongate magnets, thereby facilitating the rotation of the first and second elongate magnets into optimal alignment with the magnetic field of an axially magnetized headpiece magnet. This results in superior retention force as well as better alignment of the headpiece and implant magnets and, accordingly, better alignment of the headpiece and implant antennas. In other implementations, where the first and second magnets are non-rotatable (and may be either elongate or non-elongate) and the third elongate magnet is rotatable, the first and second magnets may be used to increase the attraction force between the magnet apparatus and the headpiece magnet, to maintain the rotatable magnet in an axial orientation, and to properly align the headpiece magnet with the magnet apparatus.

It should also be noted here that the use of an axially magnetized headpiece magnet, which is facilitated by the reduction in magnetic attraction between the two rotatable magnets associated with some implementations, is more magnetically efficient that a diametrically magnetized headpiece magnet due to the orientation of the magnetic field of the axially magnetized headpiece magnet. As a result, less magnetic material may be employed within a magnet assembly, there is less friction between rotating magnets and the inner surface of the case and a corresponding reduction in the torque associated with placement of the magnet apparatus into an MRI magnetic field as well as less of an MRI artifact.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of the exemplary embodiments will be made with reference to the accompanying drawings.

FIG. 1 is a perspective view of an implant magnet apparatus (or “magnet assembly”) in accordance with one embodiment of a present invention.

FIG. 2 is a perspective view of a portion of the implant magnet apparatus illustrated in FIG. 1.

FIG. 3 is an exploded perspective view of the implant magnet apparatus illustrated in FIG. 1.

FIG. 4 is a plan view of a portion of the implant magnet apparatus illustrated in FIG. 1.

FIG. 5 is an exploded perspective view of a portion of the implant magnet apparatus illustrated in FIG. 1.

FIG. 6 is a section view taken along line 6-6 in FIG. 1.

FIG. 7 is an enlarged view of a portion of the section view illustrated in FIG. 6.

FIG. 8 is a partial section view of a system including a headpiece and an implant with the magnet apparatus illustrated in FIG. 1.

FIG. 9 is a section view similar to FIG. 6 with the implant in an MRI magnetic field.

FIG. 10 is a perspective view of an implant magnet apparatus (or “magnet assembly”) in accordance with one embodiment of a present invention.

FIG. 11 is a perspective view of a portion of the implant magnet apparatus illustrated in FIG. 10.

FIG. 12 is an exploded perspective view of the implant magnet apparatus illustrated in FIG. 10.

FIG. 13 is a plan view of a portion of the implant magnet apparatus illustrated in FIG. 10.

FIG. 14 is a section view taken along line 14-14 in FIG. 10.

FIG. 15 is a partial section view of a system including a headpiece and an implant with the magnet apparatus illustrated in FIG. 10.

FIG. 16 is a section view similar to FIG. 14 with the implant in an MRI magnetic field.

FIG. 17 is a perspective view of an implant magnet apparatus (or “magnet assembly”) in accordance with one embodiment of a present invention.

FIG. 18 is a perspective view of a portion of the implant magnet apparatus illustrated in FIG. 17.

FIG. 19 is an exploded perspective view of the implant magnet apparatus illustrated in FIG. 17.

FIG. 20 is a plan view of a portion of the implant magnet apparatus illustrated in FIG. 17.

FIG. 21 is enlarged plan view of a portion of the implant magnet apparatus illustrated in FIG. 17.

FIG. 22 is a section view taken along line 22-22 in FIG. 17.

FIG. 23 is a partial section view of a system including a headpiece and an implant with the magnet apparatus illustrated in FIG. 17.

FIG. 24 is a section view similar to FIG. 22 with the implant in an MRI magnetic field.

FIG. 24A is an exploded perspective view of an implant magnet apparatus (or “magnet assembly”) in accordance with one embodiment of a present invention.

FIG. 24B is a section view taken along line 24B-24B in FIG. 24A.

FIG. 24C is a section view of the implant magnet apparatus illustrated in FIG. 24A.

FIG. 25 is a top view of a cochlear implant in accordance with one embodiment of a present invention.

FIG. 26 is a block diagram of a cochlear implant system in accordance with one embodiment of a present invention.

FIG. 27 is a flow chart showing a method in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

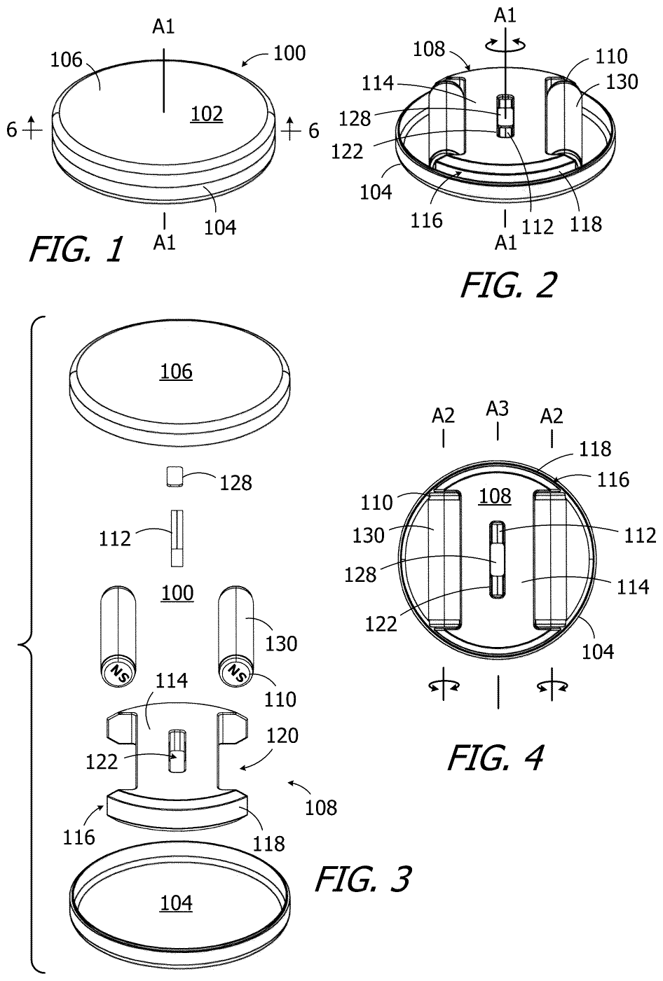

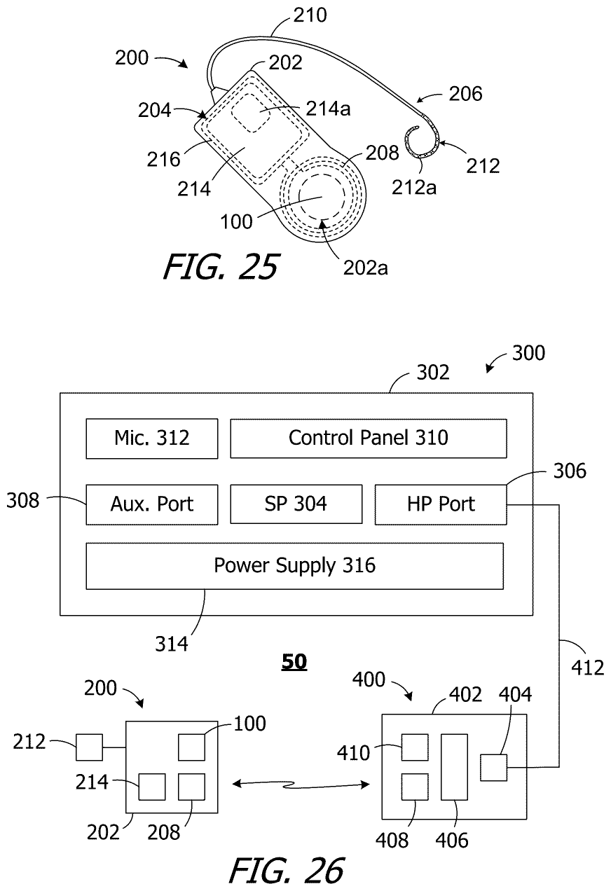

As illustrated for example in FIGS. 1-6, an exemplary magnet apparatus (or “magnet assembly”) 100 includes a case 102, with base 104 and a cover 106, a frame 108 that is rotatable relative to the case, and first and second elongate diametrically magnetized magnets 110 that are rotatable relative to the frame. In the illustrated embodiment, there are only two (i.e., two and no more than two) magnets 110. A third elongate magnet 112 is located between the first and second magnets 110, is not rotatable relative to the frame, has a N-S direction perpendicular to its longitudinal axis, and is fixed in a predetermined N-S orientation relative to the frame 108 and the remainder of the magnet assembly 100. In particular, the elongate magnet 112 is fixed in the N-S orientation illustrated in FIGS. 1-6. The magnet assembly 100 may, in some instances, be employed in a system 50 (FIG. 8) that includes a cochlear implant 200 (described below with reference to FIG. 25) with a magnet assembly 100 and an external device such as a headpiece 400 (described below with reference to FIGS. 8 and 26).

The case 102 in the exemplary magnet apparatus 100 is disk-shaped and defines a central axis A1, which is also the central axis of the frame 108. The frame 108 is rotatable relative to the case 102 about the central axis A1 over 360°. The magnets 110 and 112 rotate with the frame 108 about the central axis A1. Each magnet 110 is also rotatable relative to the frame 108 about its own longitudinal axis A2 (also referred to as “axis A2”) over 360°. The magnet 112, on the other hand, is not rotatable relative to the frame 108 about its own longitudinal axis A3. In the exemplary implementation illustrated in FIGS. 1-6, the longitudinal axes A2 and A3 are parallel to one another and are perpendicular to the central axis A1. In other implementations, the magnets 110 and/or 112 may be oriented such that the longitudinal axes thereof are at least substantially perpendicular to the central axis A1. As used herein, an axis that is “at least substantially perpendicular to the central axis” includes axes that are perpendicular to the central axis as well as axes that are slightly non-perpendicular to the central axis (i.e., axes that are offset from perpendicular by up to 5 degrees).

The exemplary case 102 is not limited to any particular configuration, size or shape. In the illustrated implementation, the case 102 is a two-part structure that includes the base 104 and the cover 106 which are secured to one another in such a manner that a hermetic seal is formed between the cover and the base. Suitable techniques for securing the cover 106 to the base 104 include, for example, seam welding with a laser welder. With respect to materials, the case 102 may be formed from biocompatible paramagnetic metals, such as titanium or titanium alloys, and/or biocompatible non-magnetic plastics such as polyether ether ketone (PEEK), low-density polyethylene (LDPE), high-density polyethylene (HDPE), ultra-high-molecular-weight polyethylene (UHMWPE), polytetrafluoroethylene (PTFE) and polyamide. In particular, exemplary metals include commercially pure titanium (e.g., Grade 2) and the titanium alloy Ti-6Al-4V (Grade 5), while exemplary metal thicknesses may range from 0.20 mm to 0.25 mm. With respect to size and shape, the case 102 may have an overall size and shape similar to that of conventional cochlear implant magnets so that the magnet apparatus 100 can be substituted for a conventional magnet in an otherwise conventional cochlear implant. The case 102 may also have an overall size and shape that is larger than that of conventional cochlear implant magnets in other embodiments. In some implementations, the diameter that may range from 9 mm to 17.4 mm and the thickness may range from 1.5 mm to 4.0 mm. The diameter of the case 102 in the illustrated embodiment is about 15 mm to about 16.0 mm and the thickness is about 3.0 mm to 4.0 mm. As used herein in the context of the case 102, the word “about”means ±10%.

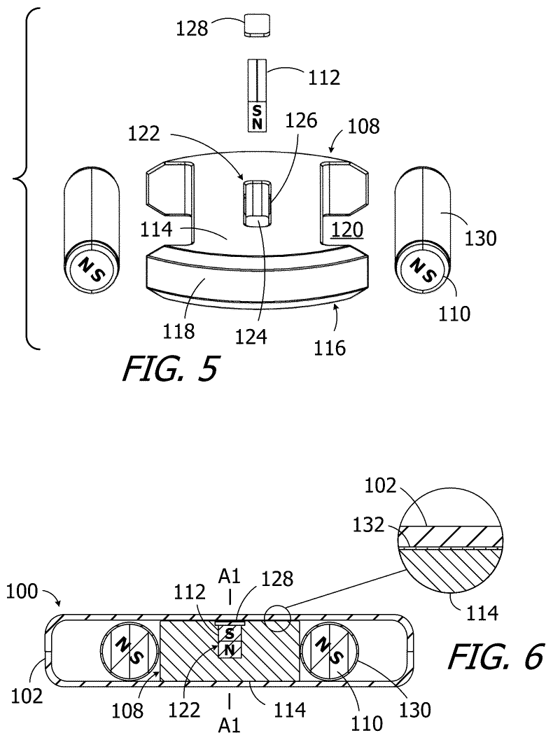

The exemplary frame 108 includes a central base member 114 and first and second end members 116, which have curved ends 118 and are located at opposite ends of the base member 114. The base member 114 and end members define receptacles 120 for the magnets 110 and a receptacle 122 for the magnet 112. Referring more specifically to FIG. 5, the exemplary receptacle 122 includes an end wall 124 and a rim 126. A cover 128 (e.g., a titanium cover) may be positioned over the magnet 112 and secured (e.g., by welding, epoxy or adhesive) to the rim 126 to hold the magnet within the receptacle 122. The cover 128 may extend over the entire length of the magnet 112, or over only a portion of the length (as shown). Alternatively, the magnet 112 may be embedded within the frame 108.

Suitable materials for the frame 108, which may be formed by machining, metal injection molding or injection molding, include paramagnetic metals, polymers and plastics such as those discussed above in the context of the case 102. Referring more specifically to FIG. 4, there may be a relatively tight fit between the between the magnets 110 and 112 and the receptacles 120 and 122. For example, the length of the receptacles 120 and 122 may be about 0.05 mm to about 0.20 mm greater than the length of the magnets 110 and 112 in some implementations. As used herein in the context of the frame, the word “about” means ±10%.

The magnets 110 in the exemplary magnet apparatus 100 are elongate diametrically magnetized magnets, and there are only two magnets 110 within the case 102. The exemplary magnets 110 are circular in a cross-section that is perpendicular to the longitudinal axis A2 and, in some instances, may have rounded corners. Although not so limited, the magnet 112 in exemplary embodiment is rectangular in a cross-section that is perpendicular to the longitudinal axis A3. Suitable materials for the magnets 110 and 112 include, but are not limited to, neodymium-boron-iron and samarium-cobalt. In the illustrated embodiment, the magnets 110 and 112 are N52 or N55 neodymium-boron-iron magnets. The length of the magnets 110 may range from about 5.0 mm to about 12.0 mm and is about 8.3 mm in the illustrated embodiment, while the diameter of the magnets 110 may range from about 1.5 mm to about 2.4 mm and is about 2.4 mm in the illustrated embodiment.

The magnet 112 in the exemplary embedment is smaller than the magnets 110. The length of the magnet 112 may range from about 3.0 mm to about 9.0 mm and is about 4.5 mm in the illustrated embodiment, the height (in the N-S direction) may range from about 1.0 mm to about 1.8 mm and is about 1.6 mm in the illustrated embodiment, and the width (perpendicular to the N-S direction) may range from about 1.0 mm to about 1.5 mm and is about 1.0 mm in the illustrated embodiment. Put another way, the volume of the magnet 112 may range from about 15% to about 30% of the volume of one of the magnets 110, and is about 18% of the volume of one of the magnets 110 in the illustrated embodiment. As used herein in the context of the magnet size, the word “about” means ±10%. The frame 108 maintains the maintains the spacing between the magnets 110, as well as between the magnets 110 and the magnet 112.

The magnets 110 may be located within tubes 130 formed from low friction material. Suitable materials for the tubes 130 include polymers, such as silicone, PEEK and other plastics, PTFE, and PEEK-PTFE blends, and paramagnet metals. The magnets 110 may be secured to the tubes 130 such that each tube rotates with the associated magnet about its axis A2, or the magnets may be free to rotate relative to the tubes. The magnet/tube combination is also more mechanically robust than a magnet alone. The magnets 110 may, in place of the tubes 130, be coated with the lubricious materials discussed below.

Friction may be further reduced by coating the inner surfaces of the case 102 and/or the surfaces of the frame 108 with a lubricious layer. The lubricious layer may be in the form of a specific finish of the surface that reduces friction, as compared to an unfinished surface, or may be a coating of a lubricious material such as diamond-like carbon (DLC), titanium nitride (TiN), PTFE, polyethylene glycol (PEG), Parylene, fluorinated ethylene propylene (FEP) and electroless nickel sold under the tradenames Nedox® and Nedox PF™. The DLC coating, for example, may be only 0.5 to 5 microns thick. In those instances where the base 104 and a cover 106 are formed by stamping, the finishing process may occur prior to stamping. Micro-balls, biocompatible oils and lubricating powders may also be added to the interior of the case to reduce friction. In the illustrated implementation, the surfaces of the frame 108 may be coated with a lubricious layer 132 (e.g., DLC), while the inner surfaces of the case 102 do not include a lubricious layer, as shown in FIG. 6. The lubricious layer 132 reduces friction between the case 102 and frame 108.

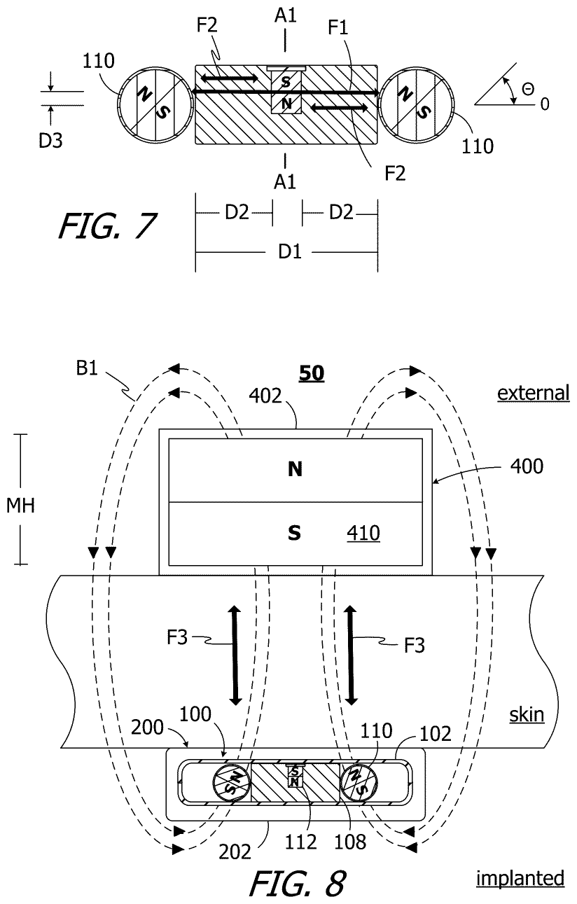

Referring to FIG. 7, which shows the orientation of the magnets 110 absent a dominant magnetic field, the distance D1 between the magnets 110 may range from about 3.8 mm to about 5.0 mm and is about 4.0 mm in the illustrated embodiment, while the distance D2 between the between the magnets 110 and the magnet 112 may range from about 1.2 mm to about 1.6 mm and is about 1.5 mm in the illustrated embodiment. As used herein in the context of the magnet spacing, the word “about” means ±10%. Absent the magnet 112, the magnets 110 would align with one another S-N-S-N horizontally in the illustrated orientation (i.e., parallel to the implant antenna plane and parallel to a plane perpendicular to axis A1) due to the magnetic force F1, despite the fact the magnetic force F1 is relatively weak as a result of the relatively large distance D1. The presence of magnet 112 and the associated magnetic forces F2 between the magnet 112 and the magnets 110, as well as the orientation of the N-S direction of the magnet 112 being the same as the axis A1 direction (i.e., perpendicular to the implant antenna plane and perpendicular to a plane perpendicular to axis A1), results in the magnets 110 each being rotationally offset from a plane perpendicular to axis A1 by an angle ⊖ that ranges from about 20° to about 45° in the absence of a dominant magnetic field. The magnet 112 also centralizes the magnetic field of the magnet apparatus 100. In other words, the magnets 110 rotate toward the magnet 112, and strongest portion the magnetic fields of the magnets 110 is focused toward the center of the headpiece magnet.

Turning to FIG. 8, the exemplary magnet apparatus (or “magnet assembly”) 100 may be part of an implanted cochlear implant 200 (described in detail below with reference to FIG. 25), with a housing 202, that is employed in conjunction with an external device such as a headpiece 400 (described in detail below with reference to FIG. 26) in a system 50. The cochlear implant 200 and headpiece 400 are shown on opposite sides of a relatively thick (i.e., 10 mm or more) skin flap. The exemplary headpiece 400 includes, among other things, a housing 402 and an axially magnetized disk-shaped positioning magnet (or “external magnet”) 410. The N-S direction of the external magnet 410 is at least substantially perpendicular (i.e., is perpendicular ±5%) to the implant recipient's skin. Although not so limited, the exemplary axially magnetized magnet 410 may have a height MH of about 7.6 mm and a diameter of about 11.45 mm. It should also be noted that although the implant 200 and headpiece 400 are configured such that like poles of the headpiece magnet 410 and the implant magnet 112 face one another (e.g., S-S as shown) across the skin, the resulting repulsive force is relatively weak given the relatively small size of magnet 112.

The respective configurations of the magnet assembly 100 and the headpiece 400 are such that when the implanted magnets 110 are exposed to the magnetic field B1 of the axially magnetized external magnet 410 across the relatively thick skin flap, the magnetic attraction force F3 between the external magnet 410 and an implanted magnet 110 is greater than the sum of the magnetic attraction force F1 between the two magnets 110 and the magnetic attraction force F2 between that magnet 110 and the magnet 112. The magnetic attraction force F3 may be, for example, at least 10% greater than the combined magnetic attraction forces F1+F2, or may be, for example, at least 20% greater than the combined magnetic attraction forces F1+F2. As a result, the magnets 110 advantageously rotate from the state illustrated in FIGS. 1-7 into alignment with the magnetic field B1 of the axially magnetized external magnet 410, as shown in FIG. 8. Put another way, the individual magnetic dipole moments of the elongate diametrically magnetized implant magnets 110 are oriented substantially in the direction of the magnetic field of the axially magnetized external magnet 410 during attractive transcutaneous magnetic interaction with the axially magnetized external magnet 410. The axially magnetized magnet 410 will also align with the center of the magnet apparatus 100, thereby aligning the headpiece antenna with the implant antenna. The magnets 110 will return to the state illustrated in FIGS. 1-7, due to the presence of the magnet 112, when the headpiece 400 and the associated magnetic field B1 is removed.

Another aspect of the exemplary magnet apparatus 100 is the impact resistance associated with the locations of the elongate diametrically magnetized magnets 110. When the magnet apparatus 100 is subjected to an impact force (e.g., when the user bumps his/her head), the central portion of the case 102 will deflect inwardly. Advantageously, the magnets 110 are offset from the central axis A1 of the case 102 by one-half of the distance D1 (FIG. 7), which reduces the likelihood of damage to the magnets as compared to a similar magnet apparatus where at least some of the magnets are located at or near the central axis A1. The magnet 112 is protected by the frame 108.

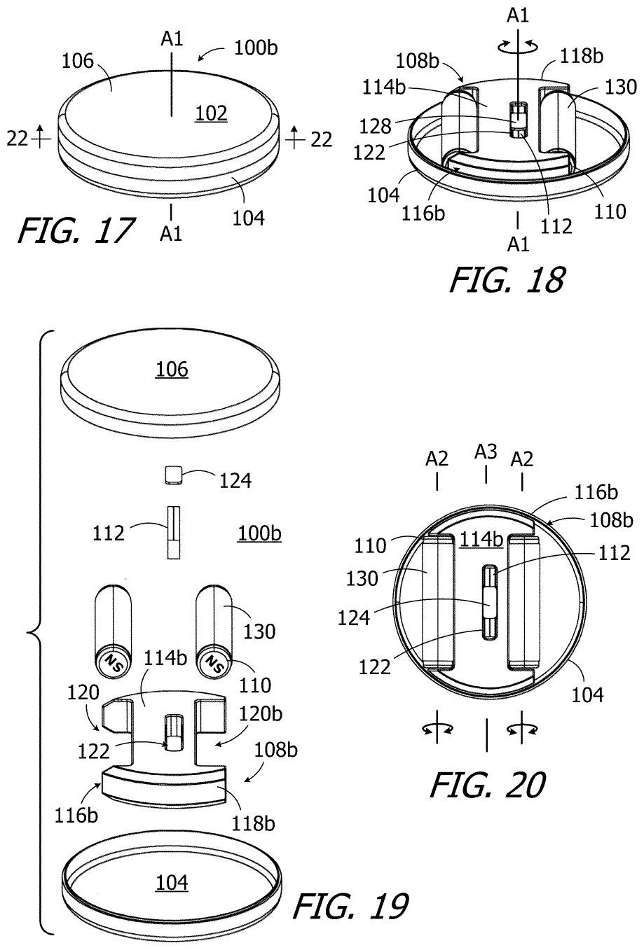

It should be noted here that although the diametrically magnetized magnets 110 are identical to one another, are parallel to one another, and are equidistant from the central axis A1 of the case 102 in the illustrated embodiment, the present magnet apparatus are no so limited. By way of example, but not limitation, the diametrically magnetized magnets 110 may have different lengths and/or may have different diameters and/or may be formed from materials having the same or different strength. Alternatively, or in addition, the diametrically magnetized magnets 110 may be non-parallel. Alternatively, or in addition, the diametrically magnetized magnets 110 may be different distances from the central axis A1 of the case 102, as discussed below with reference to FIGS. 17-22, with the configurations of the receptacles 120 adjusted to accommodate these differences.

The magnet 112, as well as the relationships between the magnet 112 and the other aspects of the exemplary magnet apparatus 100, are not limited to those described above. By way of example, but not limitation, the N-S center of the magnet 112 is offset from centers of the magnets 110 (i.e., is offset from the plane defined by the axes A2) by as distance D3 (FIG. 7). The distance D3 is about 0.2 mm in the illustrated embodiment and may be increased or decreased to adjust the manner in which the magnets 110 interact with the headpiece magnet 410 or other headpiece magnets. Alternatively, or in addition, the size, shape and/or volume of the magnet 112 may be varied. For example, the length may be increased, and the width may be decreased. A longer magnet 112 will reduce the interaction between the magnets 110, as compared to a shorter magnet 112.

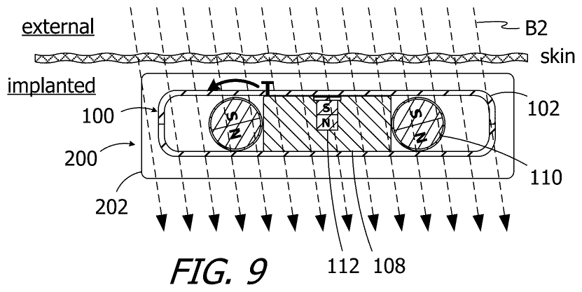

In any case, when exposed to a dominant MRI magnetic field B2 (FIG. 9), the torque T on the magnets 110 will rotate the magnets about their axis A2 (FIG. 4), thereby aligning the magnetic fields of the magnets 110 with the MRI magnetic field B2. Although magnet 112 does not rotate, the associated torque is minimal torque because magnet 112 is small. The frame 108 will also rotate about axis A1 as necessary to align the magnetic fields of the magnets 110 and 112 with the MRI magnetic field B2. When the magnet apparatus 100 is removed from the MRI magnetic field B2, the magnetic attraction between the magnets 110 will cause the magnets 110 to rotate about axis A2 back to the orientation illustrated in FIGS. 1-7.

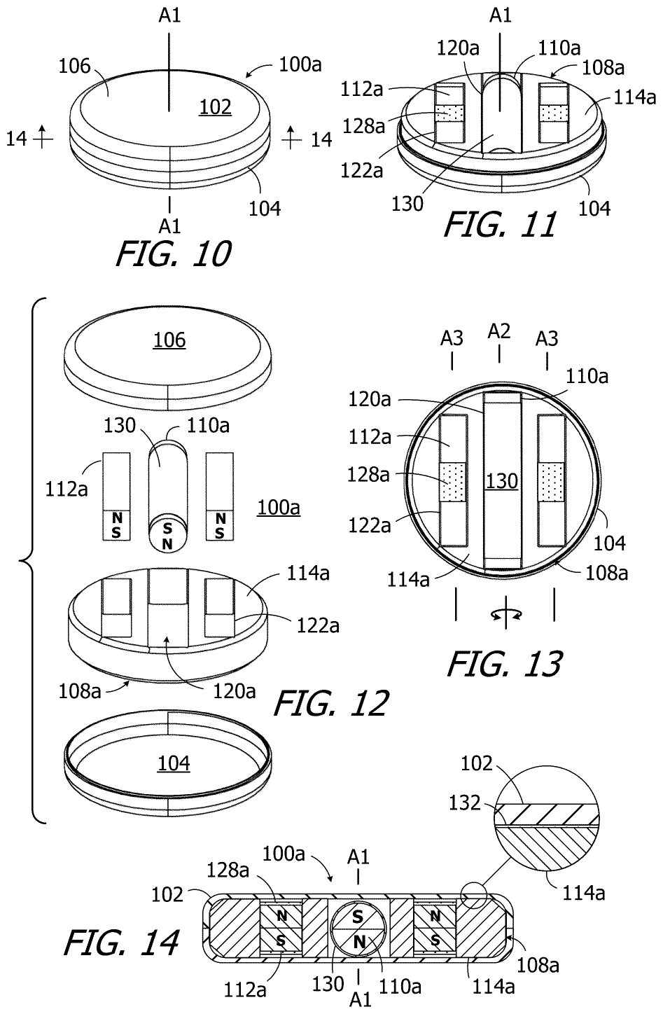

Another exemplary magnet apparatus (or “magnet assembly”) is generally represented by reference numeral 100a in FIGS. 10-14. Magnet apparatus 100a is substantially similar to magnet apparatus 100 and similar elements are represented by similar reference numerals. For example, the magnet apparatus 100a includes a case 102, with a base 104 and a cover 106, a rotatable frame 108a with a lubricious layer 132. Here, however, there is only one (i.e., one and more than one) magnet 110a that is rotatable about its longitudinal axis A2 and only two magnets 112a that are not rotatable about their longitudinal axes A3. The frame 108a includes a disk 114a, a single receptacle 120a for the magnet 110a, and a pair of receptacles 122a for the magnets 112a.

The rotatable magnet 110a in the exemplary magnet apparatus 100a is elongate diametrically magnetized magnet that is circular in cross-section perpendicular to the axis A2. The rotatable magnet 110a may be longer, due to its location within the frame 108a, than the rotatable magnets 110 in a magnet apparatus 100a that is the same overall size as the magnet apparatus 100. The exemplary non-rotatable magnets 112a are rectangular in cross-sections perpendicular to their axes A3 and the N-S directions of the magnets 112a are perpendicular to the axis A1 in the illustrated implementation. The non-rotatable magnets 112a may, however, be other shapes, may have other N-S orientations, and/or there may be more than two non-rotatable magnets in other implementations. For example, each of the illustrated non-rotatable magnets 112a may be replaced by one or more axially magnetized disc-shaped magnets (not shown). Alternatively, or in addition, the N-S direction of the non-rotatable magnets may be oriented at an angle relative to axis A1 that is not parallel or perpendicular to axis A1 (e.g., 45° relative to axis A1). Suitable magnetic materials for the magnets 110a and 112a are described above with reference to magnets 110 and 112. It should be noted, however, that magnets 112a are relatively weak either by virtue of magnetic material such as that is employed (as shown) or a small size (in other embodiments). For example, the volume of the non-rotatable magnets 112a may be about 30% to 60% of the volume of the rotatable magnet 110a. Depending on size, suitable magnetic materials include N35-N42 and N52-N55 neodymium-boron-iron magnets.

The magnets 112a may be secured to the frame 108a and within the receptacles 122a in any suitable manner. In the illustrated embodiment, the magnets 112a are secured with adhesive 128a. In other implementations, a cover (e.g., a titanium cover) may be positioned over each magnet 112a and secured (e.g., by welding, epoxy or adhesive) to the rim of the receptacle 122a. The adhesive or cover may extend over the entire length of the magnet 112a, or over only a portion of the length. The magnets 112a may also be embedded within the frame 108a.

The exemplary magnet assembly 100a is shown in the absence of a dominant magnetic field in FIGS. 10-14. Here, and referring to FIG. 14, the south pole of the magnet 110a is aligned with the north poles of the magnets 112a, and the north pole of the magnet 110 is aligned with the south poles of the magnets 112a.

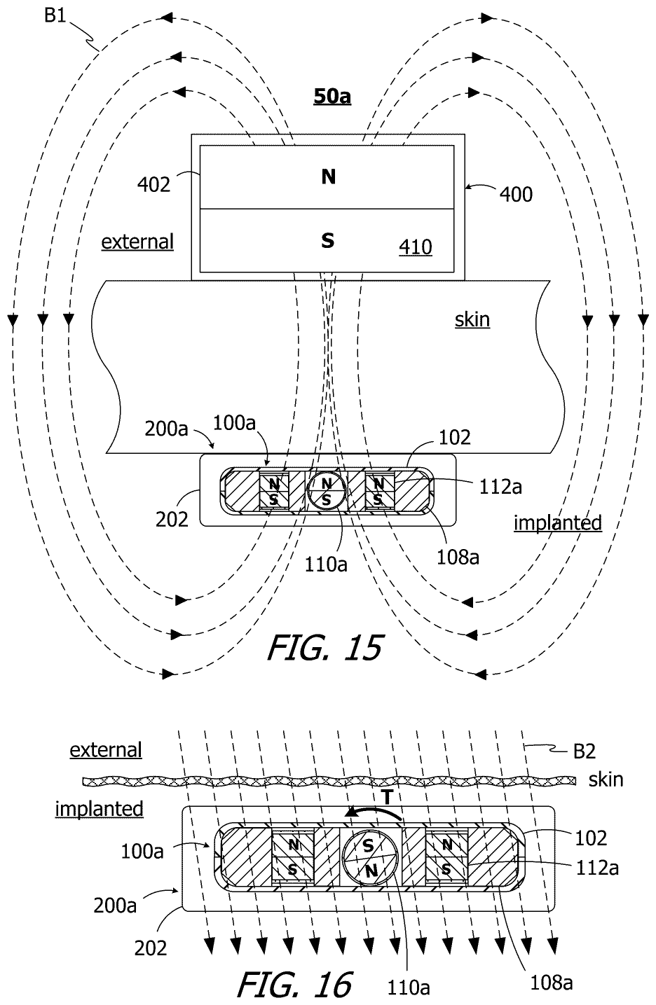

Turning to FIG. 15, the exemplary magnet apparatus (or “magnet assembly”) 100a may be part of an implanted cochlear implant 200a that is otherwise identical to cochlear implant 200 and that is employed in conjunction with an external device such as the above-described headpiece 400 in a system 50a. here too, the cochlear implant 200a and headpiece 400 are shown on opposite sides of a relatively thick (i.e., 10 mm or more) skin flap and the implant 200a and headpiece 400 are configured such that opposite poles of the headpiece magnet 410 and the mechanically implant magnets 110a and 112a face one another (e.g., N-S as shown) across the skin.

The respective configurations of the magnet assembly 100a and the headpiece 400 are such that when the implanted magnet 110a is exposed to the magnetic field B1 of the axially magnetized external magnet 410 across the relatively thick skin flap, the magnetic attraction force between the external magnet 410 and an implanted magnet 110a is greater (e.g., at least 10% greater or at least 20% greater) than the magnetic attraction forces between the magnet 110a and the two magnets 112a. As a result, the magnet 110a advantageously rotates from the state illustrated in FIG. 14 into alignment with the magnetic field B1 of the axially magnetized external magnet 410, as shown in FIG. 15. Put another way, the magnetic dipole moment of the elongate diametrically magnetized implant magnet 110a is oriented substantially in the direction of the magnetic field of the axially magnetized external magnet 410 during attractive transcutaneous magnetic interaction with the axially magnetized external magnet 410. The axially magnetized magnet 410 will also align with the center of the magnet apparatus 100a, thereby aligning the headpiece antenna with the implant antenna. The magnet 110a will return to the state illustrated in FIG. 14, due to the presence of the magnets 112a, when the headpiece 400 and the associated magnetic field B1 is removed.

When exposed to a dominant MRI magnetic field B2 in the manner illustrated FIG. 16, the torque T on the magnet 110a will rotate the magnet about its axis A2 (FIG. 13), thereby aligning the magnetic field of the magnet 110a with the MRI magnetic field B2. Although magnets 112a do not rotate, the associated torque is minimal torque because magnet 112a is relatively weak. The frame 108a will also rotate about axis A1 as necessary to align the magnetic fields of the magnet 110a and 112a with the MRI magnetic field B2. When the magnet apparatus 100a is removed from the MRI magnetic field B2, the magnetic attraction between the magnet 110a and the magnets 112a will cause the magnet 110a to rotate about axis A2 back to the orientation illustrated in FIG. 14.

Another exemplary magnet apparatus (or “magnet assembly”) is generally represented by reference numeral 100b in FIGS. 17-22. Magnet apparatus 100b is substantially similar to magnet apparatus 100 and similar elements are represented by similar reference numerals. For example, the magnet apparatus 100b includes a case 102, with a base 104 and a cover 106, a rotatable frame 108b with a lubricious layer 132, only two magnets 110 that are rotatable about their longitudinal axes A2, and a magnet 112 that is not rotatable about its longitudinal axis A3. The frame 108b includes a base member 114b and first and second end members 116b, receptacles 120 and 120b for the magnets 110, and a receptacle 122 for the magnet 112. The magnet 112 prevents the magnets 110 from aligning with one another S-N-S-N horizontally in the illustrated orientation (i.e., parallel to the implant antenna plane and parallel to a plane perpendicular to axis A1) as is described above. Here, however, the internal components of the magnet apparatus are asymmetric. For example, one of the rotatable magnets 110 may be closer to the non-rotatable magnet 112 than the other. As a result, an effect similar to that described above with reference to magnet apparatus 100 may be obtained with a non-rotatable magnet 112 that is weaker (due to size and/or choice of magnetic material) than the non-rotatable magnet 112 in magnet apparatus 100. The weaker magnet 112 reduces the associated headpiece magnet repulsion as well as the torque associated with an MRI magnet field.

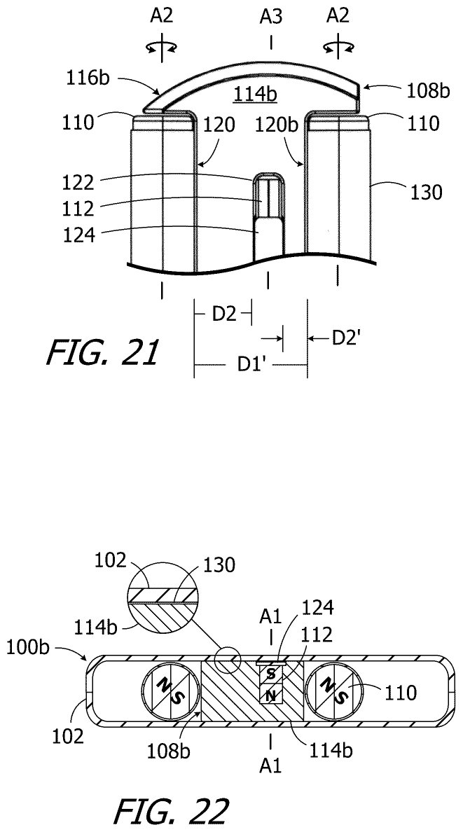

Referring more specifically to FIG. 21, the magnets 110 and receptacles 120 and 120b in the exemplary magnet apparatus 100b are asymmetrically located. The distance D2 between the magnet 110 in the receptacle 120 and the magnet 112 is the same as that described above with reference to magnet apparatus 100 (FIG. 7), i.e., the distance D2 may range from about 1.2 mm to about 1.6 mm and is about 1.5 mm in the illustrated embodiment. The distance D2′ between the magnet 110 in the receptacle 120b and the magnet 112 is less than the distance D2 and may range from about 0.5 mm to about 1.2 mm and is about 1.0 mm in the illustrated embodiment. The distance D1′ between the magnets 110 is correspondingly less than the distance D1 described above with reference to magnet apparatus 100 (FIG. 7). The distance D1′ may range from about 3.5 mm to about 5.0 mm and is about 4.4 mm in the illustrated embodiment. As used herein in the context of the magnet spacing, the word “about” means ±10%. The materials and other dimensions of the magnet apparatus 100b may be the same as those described above with reference to the magnet apparatus 100.

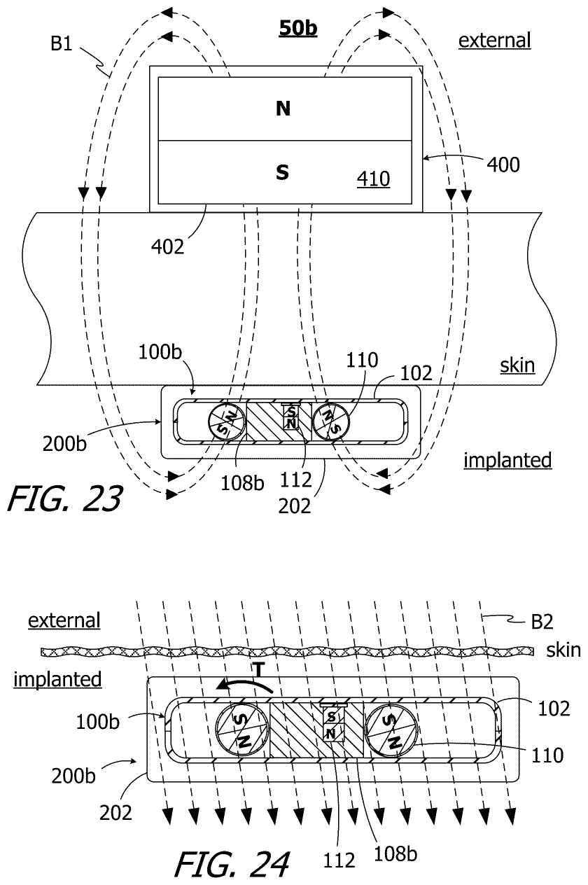

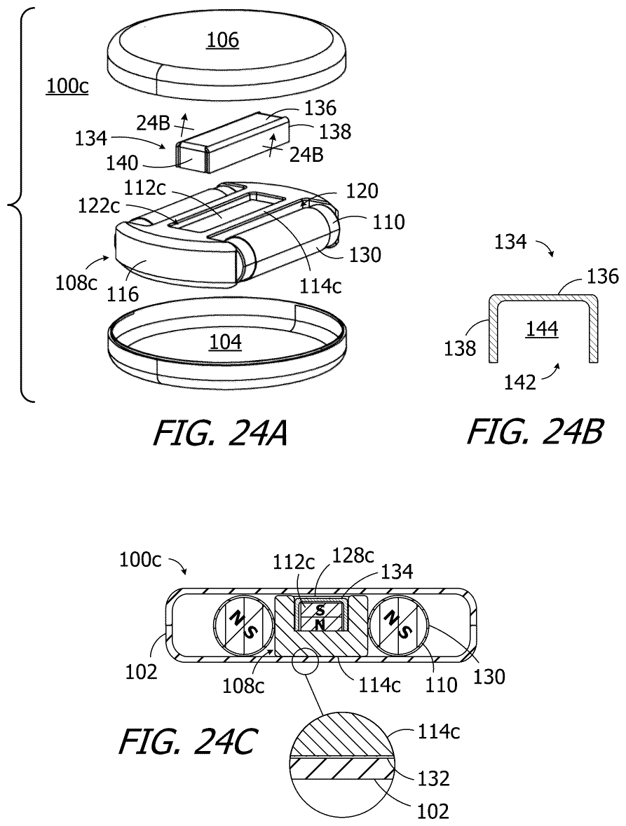

As illustrated for example in FIG. 22, the orientation of the magnets 110 in magnet apparatus 100b absent a dominant magnetic field is essentially the same as that described above with reference to magnet apparatus 100. Turning to FIG. 23, the respective configurations of the magnet assembly 100b and the headpiece 400 are such that when the implanted magnets 110 are exposed to the magnetic field B1 of the axially magnetized external magnet 410 across the relatively thick skin flap, the magnetic attraction force between the external magnet 410 and each implanted magnet 110 is greater (e.g., at least 10% greater or at least 20% greater) than the sum of the magnetic attraction force between the two magnets 110 and the magnetic attraction force between each magnet 110 and the magnet 112. As a result, the magnets 110 advantageously rotate from the state illustrated in FIG. 22 into alignment with the magnetic field B1 of the axially magnetized external magnet 410, as shown in FIG. 23. Similarly, as illustrated for example in FIG. 24, the magnets 110 in magnet apparatus 100b will rotate into alignment with an MRI magnetic field B2 in the manner described above with reference to FIG. 9.

Another exemplary magnet apparatus (or “magnet assembly”) is generally represented by reference numeral 100c in FIGS. 24A-24C. Magnet apparatus 100c is substantially similar to magnet apparatus 100 and similar elements are represented by similar reference numerals. For example, the magnet apparatus 100c includes a case 102 with a base 104 and a cover 106, a rotatable frame 108c with a lubricious layer 132, only two magnets 110, which are rotatable about their longitudinal axes, and a magnet 112c that is not rotatable about its longitudinal axis. The frame 108c includes a base member 114c and first and second end members 116, receptacles 120 for the magnets 110, and a receptacle 122c for the magnet 112c. The magnet 112c prevents the magnets 110 from aligning with one another S-N-S-N horizontally in the illustrated orientation (i.e., parallel to the implant antenna plane and parallel to a plane perpendicular to axis A1) as is described above.

Here, however, the magnet 112c is at least partially covered by a magnetic shield (or “shield”) 134 that focuses the magnetic field away from the patient's skin and the associated headpiece magnet. The rotatable magnets 110, on the other hand, are not covered by the shield 134 or any other magnetic field focusing shield. The shield 134 reduces the repulsive magnetic force between the magnet 112c and the headpiece magnet and, accordingly, improves headpiece retention. The rotational orientations of the rotatable magnets 110 are influenced by the magnet 112c, due to the close proximity thereof, as well as by the magnetic shield 134. In the illustrated implementation, the shield 134 includes a top portion (or headpiece-facing portion) 136, longitudinally extending side portions 138, end portions 140, an open bottom (or headpiece-facing portion) 142, and an internal volume 144 in which the magnet 112c located. In other implementations, the bottom portion may be closed and/or the end portions may be omitted. Suitable materials for the shield 134 include, but are not limited to, iron or a nickel-iron alloy, referred to as mu-metal, that is composed of approximately 77% nickel, 16% iron, 5% copper and 2% chromium or molybdenum.

The magnet 112c and shield 134 are located within the receptacle 122c and may be held in place with adhesive 128c. In other implementations, a cover (e.g., a titanium cover) may be positioned over the shield 134 and secured (e.g., by welding, epoxy or adhesive) to the rim of the receptacle 122c. The adhesive or cover may extend over the entire length of the shield 134, or over only a portion of the length. The magnet 112c and shield 134 may also be embedded within the frame 108c.

One example of a cochlear implant (or “implantable cochlear stimulator”) including the present magnet apparatus 100 (or 100a or 100b or 100c) is the cochlear implant 200 illustrated in FIG. 25. The cochlear implant 200 includes a flexible housing 202 formed from a silicone elastomer or other suitable material, a processor assembly 204, a cochlear lead 206, and an antenna 208 that may be used to receive data and power by way of an external antenna that is associated with, for example, a sound processor unit. The cochlear lead 206 may include a flexible body 210, an electrode array 212 at one end of the flexible body, and a plurality of wires (not shown) that extend through the flexible body from the electrodes 212a (e.g., platinum electrodes) in the array 212 to the other end of the flexible body. The magnet apparatus 100 is located within a region encircled by the antenna 208 (e.g., within an internal pocket 202a defined by the housing 202) and ensures that an external antenna (discussed below) will be properly positioned relative to the antenna 208. The exemplary processor assembly 204, which is connected to the electrode array 212 and antenna 208, includes a printed circuit board 214 with a stimulation processor 214a that is located within a hermetically sealed case 216. The stimulation processor 214a converts the stimulation data into stimulation signals that stimulate the electrodes 212a of the electrode array 212.

Turning to FIG. 26, the exemplary cochlear implant system 50 includes the cochlear implant 200, a sound processor, such as the illustrated body worn sound processor 300 or a behind-the-ear sound processor, and a headpiece 400.

The exemplary body worn sound processor 300 in the exemplary ICS system 50 includes a housing 302 in which and/or on which various components are supported. Such components may include, but are not limited to, sound processor circuitry 304, a headpiece port 306, an auxiliary device port 308 for an auxiliary device such as a mobile phone or a music player, a control panel 310, one or more microphones 312, and a power supply receptacle 314 for a removable battery or other removable power supply 316 (e.g., rechargeable and disposable batteries or other electrochemical cells). The sound processor circuitry 304 converts electrical signals from the microphone 312 into stimulation data. The exemplary headpiece 400 includes a housing 402 and various components, e.g., a RF connector 404, a microphone 406, an antenna (or other transmitter) 408 and an axially magnetized disk-shaped positioning magnet 410, that are carried by the housing. The headpiece 400 may be connected to the sound processor headpiece port 306 by a cable 412. The external positioning magnet 410 is attracted to the magnet apparatus 100 of the cochlear stimulator 200 (see FIG. 8), thereby aligning the antenna 408 with the antenna 208. The stimulation data and, in many instances power, is supplied to the headpiece 400. The headpiece 400 transcutaneously transmits the stimulation data, and in many instances power, to the cochlear implant 200 by way of a wireless link between the antennae. The stimulation processor 214a converts the stimulation data into stimulation signals that stimulate the electrodes 212a of the electrode array 212.

In at least some implementations, the cable 412 will be configured for forward telemetry and power signals at 49 MHz and back telemetry signals at 10.7 MHz. It should be noted that, in other implementations, communication between a sound processor and a headpiece and/or auxiliary device may be accomplished through wireless communication techniques. Additionally, given the presence of the microphone(s) 312 on the sound processor 300, the microphone 406 may be also be omitted in some instances.

The functionality of the sound processor 300 and headpiece 400 may also be combined into a single head wearable sound processor that includes all of the external components (e.g., the battery, microphone, sound processor, antenna coil and magnet). Examples of head wearable sound processors are illustrated and described in U.S. Pat. Nos. 8,811,643 and 8,983,102, which are incorporated herein by reference in their entirety. Headpieces and head wearable sound processors are collectively referred to herein as “head wearable external components.”

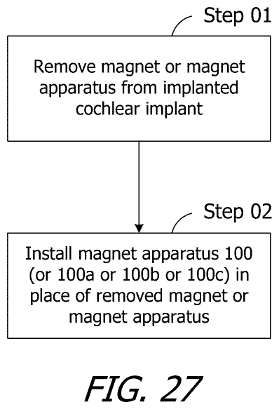

The present inventions are applicable to systems that include cochlear implants which have already been implanted into the recipient. For example, a similarly sized magnet, or a magnet apparatus with a similarly sized case, may be removed in situ from an implanted cochlear implant (Step 01) in the exemplary method illustrated in FIG. 27. In some instances, the magnet or magnet apparatus may be removed from a pocket in the cochlear implant housing. The exemplary magnet apparatus 100 (or 100a or 100b or 100c) described herein may be installed in place of the removed magnet or magnet apparatus (Step 02). In some instances, the magnet apparatus 100 (or 100a or 100b or 100c) may be inserted into the same pocket in the cochlear implant housing from which magnet or magnet apparatus was removed. Suitable removal and installation tools and techniques are illustrated and described in U.S. Pat. No. 10,124,167, which is incorporated herein by reference in its entirety. The headpiece magnet in the associated system may, if necessary, be removed from the headpiece or other head wearable external component and replaced with an axially magnetized magnet.

Although the inventions disclosed herein have been described in terms of the preferred embodiments above, numerous modifications and/or additions to the above-described preferred embodiments would be readily apparent to one skilled in the art. The inventions include any combination of the elements from the various species and embodiments disclosed in the specification that are not already described. It is intended that the scope of the present inventions extend to all such modifications and/or additions and that the scope of the present inventions is limited solely by the claims set forth below.

1. A magnet assembly, comprising:
a case defining a central axis;
a magnet frame within the case and rotatable about the central axis of the case; and
first, second and third elongate magnets that are located within the frame, with the third elongate magnet located between the first and second elongate magnets, and that each define a longitudinal axis and a N-S direction that is perpendicular to the longitudinal axis;
wherein
the first and third elongate magnets are separated from one another by a first fixed non-zero distance that is perpendicular to at least one of their longitudinal axes;
the second and third elongate magnets are separated from one another by a second fixed non-zero distance that is perpendicular to at least one of their longitudinal axes;
at least one of the elongate magnets is rotatable about its longitudinal axis relative to the frame; and
at least one of the elongate magnets is not rotatable about its longitudinal axis relative to the frame.

2. A magnet assembly as claimed in claim 1, wherein
the first fixed non-zero distance is equal to the second fixed non-zero distance.

3. A magnet assembly as claimed in claim 1, wherein
the first fixed non-zero distance is not equal to the second fixed non-zero distance.

4. A magnet assembly as claimed in claim 1, wherein
the first and second elongate magnets are rotatable about their respective longitudinal axes relative to the frame; and
the third elongate magnet is not rotatable about its longitudinal axis relative to the frame.

5. A magnet assembly as claimed in claim 4, wherein
the first and second elongate magnets define circular cross-sections in planes perpendicular to their respective longitudinal axes and are diametrically magnetized; and
the third elongate magnet defines a rectangular cross-section in a plane perpendicular to its longitudinal axes.

6. A magnet assembly as claimed in claim further comprising:
a magnetic shield located within the case and defining an internal volume;
wherein the third elongate magnet is located within the internal volume of the shield.

7. A magnet assembly as claimed in claim 1, wherein
the first and second elongate magnets are not rotatable about their respective longitudinal axes relative to the frame; and
the third elongate magnet is rotatable about its longitudinal axis relative to the frame.

8. A magnet assembly as claimed in claim 7, wherein
the first and second elongate magnets define rectangular cross-sections in planes perpendicular to their respective longitudinal axes; and
the third elongate magnet defines a circular cross-section in a plane perpendicular to its longitudinal axes and is diametrically magnetized.

9. A magnet assembly as claimed in claim 1, wherein
the magnet assembly includes only three elongate magnets.

10. A magnet assembly as claimed in claim 1, wherein
the third elongate magnet is smaller than the first elongate magnet and is smaller than the second elongate magnet.

11. A magnet assembly as claimed in claim 10, wherein
the first, second and third elongate magnets define respective volumes; and
the volume of third elongate magnet is about 15% to about 30% of the volume of the first elongate magnet and is about 15% to about 30% of the volume of the elongate second magnet.

12. A magnet assembly as claimed in claim 1, wherein
absent a dominant magnetic field, the respective N-S directions of the first and second elongate magnets are not perpendicular to the N-S direction of the third magnet.

13. A magnet assembly as claimed in claim 1, wherein
absent a dominant magnetic field, the respective N-S directions of the first and second elongate magnets are rotationally offset from a plane perpendicular to the central axis.

14. A magnet assembly as claimed in claim 1, wherein
absent a dominant magnetic field, the respective N-S directions of the first and second magnets are rotationally offset from a plane perpendicular to the central axis by about 20° to about 45°.

15. A magnet assembly, comprising:
a case;
a magnetic shield located within the case and defining an internal volume; and
a plurality of magnets located within the case;
wherein
at least one of the magnets is located within the internal volume of the shield; and
at least one of the magnets is not located within the internal volume of the shield.

16. A magnet assembly as claimed in claim 15, wherein
the magnetic shield is formed from mu-metal.

17. A magnet assembly as claimed in claim 15, wherein
the case defines a central axis;
the magnet assembly further comprises a magnet frame within the case and rotatable about the central axis of the case; and
the magnets are located within the frame and at least one of the magnets is rotatable relative to the frame.

18. A magnet assembly as claimed in claim 17, wherein
the plurality of magnets comprises first, second and third elongate magnets that define a longitudinal axis and a N-S direction that is perpendicular to the longitudinal axis;
the first and second is elongates magnets are each rotatable about its longitudinal axis relative to the frame;
the third elongate magnet is located between the first and second elongate magnets, is located within the internal volume of the shield, and is not rotatable its longitudinal axis relative to the frame.

19. A system, comprising:
a cochlear implant having
a cochlear lead including a plurality of electrodes,
an implant antenna,
an implant processor operably connected to the implant antenna and to the cochlear lead, and
a magnet assembly as claimed in claim 1 adjacent to the implant antenna; and
a head wearable external component including an axially magnetized external magnet and an external antenna adjacent to the axially magnetized external magnet.

20. A system as claimed in claim 19, wherein
the third elongate magnet and the external magnet define respective N-poles and S-poles;
the predetermined N-S rotational orientation of the third elongate magnet and the orientation of the external magnet within the external component are such that the like poles of the third elongate magnet and the external magnet face one another and are the poles that are closest to one another when the external magnet is attracted to the magnet assembly.

21. A method, comprising
removing a magnet or a magnet assembly from an implanted cochlear implant; and
installing a magnet assembly as described in claim 1 in place of the removed magnet or magnet assembly.

22. A method as claimed in claim 21, further comprising:
removing a magnet from a head wearable external component; and
replacing the magnet removed from the head wearable external component with an axially magnetized magnet.
